(19) United States
(12) Patent Application Publication
(10) Pub. No.: US 2020/0032372 A1
(43) Pub. Date: Jan. 30, 2020

(54) BIORESORBABLE IMPLANTS MADE OF EXTRUDED POWDER WITH VARYING CHEMICAL COMPOSITION

(71) Applicant: CORTRONIK GmbH, Rostock (DE)

(72) Inventors: Ullrich Bayer, Bad Doberan (DE); Jan Hannemann, Rostock (DE); Okechukwu Anopuo, Rostrock (DE)

(21) Appl. No.: 16/337,634

(22) PCT Filed: Oct. 9, 2017
(86) PCT No.: PCT/EP2017/075645

§ 371 Date: Mar. 28, 2019

(30) Foreign Application Priority Data
Oct. 10, 2016 (DE) 102016119227.9

(51) Int. Cl. C22C 1/04; C22C 33/02; A61L 31/14; A61L 31/16; A61L 31/02

(52) U.S. Cl. CPC C22C 1/0416; C22C 1/0483; C22C 33/0278; A61L 31/022; A61L 31/146; A61L 31/16; A61L 31/148

(57) ABSTRACT

The invention relates to a powder mixture for producing an alloy, a powder metallurgy process for producing a material, a material, and a medical implant made from it.

### CROSS REFERENCE TO RELATED APPLICATIONS

This is a US national phase application of PCT/EP2017/075645, filed Oct. 9, 2017, which claims priority to German patent application serial no. 102016119227.9, filed Oct. 10, 2016; the entire content of each is herein incorporated by reference in their entirety.

### TECHNICAL FIELD

The invention relates to a powder mixture for producing an alloy, a powder metallurgy process for producing a material, a material, and a medical implant.

### BACKGROUND OF THE INVENTION

In most cases, magnesium-based materials that have been produced on the basis of sintered “alloy precursors” do not achieve the mechanical properties of alloys that have been produced by metallurgical processes that involve smelting. This is attributed to the stable oxide layers surrounding each individual powder particle, which hinder or prevent both mass transfer caused by diffusion in the solidus temperature range and also the contact of molten phases among one another. These effects always occur when powdered starting materials have high surface/volume ratios and their oxides have extremely high affinity for oxygen.

The goal of using magnesium-based sintered materials produced by powder metallurgy for medical absorbable orthopedic implants is often to exploit the residual porosity that results from using this process. This can be made use of, e.g., for better growing-in behavior of bones. However, this residual porosity also has decisive functional disadvantages.

For instance, the remainders of “spacers” (e.g., urea or ammonium hydrogen carbonate), which are used for selective adjustment of the degree of porosity, cannot be completely removed without impairment of the functional properties of the implant material. This means that the pores caused by the spacers are too large to be completely closed by subsequent hot working processes. Thus, the structure of the final product still contains pores whose dimensions preclude its use as a cyclically stressed load-bearing components such as vascular stents. Therefore, reducing the negative consequences of the porosity requires time-consuming downstream sintering processes.

Furthermore, the degree of porosity per se can only vary within certain limits. If it should be kept as small as possible due to mechanical requirements on the implant, elaborate technological precautions should be taken, such as, e.g., applying a vacuum or multi-stage treatment processes under a shielding gas atmosphere.

### SUMMARY OF THE INVENTION

Starting from the above, the invention has the object of indicating an improved process for producing a material or an improved material, in particular in the form of a bioresorbable, high alloy material.

This problem is solved by a powder mixture proposed here, a process proposed here, and a resulting material. Further aspects of the invention relate to a medical implant. These implants meet the requirements both for sufficient mechanical strength and also for optimal and—depending on the application—variable chemical composition. Advantageous embodiments of the individual aspects of this invention are described below.

Accordingly, a powder mixture is proposed for production by powder metallurgy of an alloy, this powder mixture comprising or consisting of
A first metal powder selected from the group consisting of magnesium, aluminum, zinc, calcium, and iron;
At least one second metal powder different from the first metal powder selected from the group consisting of magnesium, aluminum, zinc, calcium, and iron;
At least one metal salt powder; and
Optionally at least one powder of a bioresorbable, preferably eutectic metal alloy;
Optionally at least one powder of a bioresorbable, bone-growth promoting compound from the group of the calcium phosphates.

Here the term metal powder means a pure metal that consists of one element, except for the usual impurities, and preferably has a purity of at least 99.8%.

The powder mixture proposed here makes it possible to produce alloys, in particular bioresorbable alloys, directly by power metallurgy, without the starting materials first needing to be alloyed by classic metallurgical processes that involve smelting. The powder mixture proposed here is put together in such a way that the starting metals are alloyed during the working. Putting the starting powders together in this way produces alloys that form hardly any secondary or tertiary phases, or none at all. This makes it possible to produce, from the alloy, materials and components, in particular implants, that are free of separation, which are distinguished by increased fracture-resistance, especially in the case of cyclic mechanical load when used as an implant. Alloys in specific ratios of the constituents can be produced which would show separation if produced by conventional methods such as smelting.

In a preferred embodiment of the powder mixture, the first metal powder is made of magnesium, iron, or zinc, especially magnesium. Accordingly, the powder mixture proposed here makes it possible to produce magnesium, iron, or zinc alloys.

Furthermore, selection of the type and proportion of the preferably bioresorbable metal powder (metal components) can cause wide variation in the degradation time of the material or implant under physiological conditions.

The proportion of essential, biologically active elements (such as e.g. Zn) and compounds (such as e.g. Ca phosphate) can be freely determined depending on the application (bone implant or vessel implant).

Furthermore, the powder mixture comprises at least one metal salt powder selected from the group comprising or consisting of salts of the elements lithium, sodium, potassium, magnesium, calcium, strontium, titanium, zirconium, manganese, iron, and zinc. In particular, the at least one metal salt powder is selected from the group comprising or consisting of magnesium hydrogen phosphate, magnesium carbonate, calcium phosphate, tricalcium phosphate, calcium carbonate, calcium hydroxide, calcium fluoride, lithium stearate, calcium stearate, magnesium stearate, zinc pyrophosphate, and zinc carbonate. The metal salts proposed here are bioresorbable, which makes them suitable for preparing bioresorbable alloys. Moreover, the metal salts mentioned here actively influence the special mechanical properties of the alloys. It is preferred to introduce at least two of the metal salt powders into the powder mixture proposed here. However, it is also possible to introduce more than two metal salt powders, such as, such as, for example, three, four, five, or more.

For instance, especially the use, in the starting material, of bioactive (e.g., favorably influencing bone growth) calcium and phosphorus compounds—which simultaneously also have friction-minimizing effects during subsequent hot working—can avoid contamination of the implants with technological auxiliary agents (such as, e.g., lubricants).

Furthermore, it makes it possible to avoid the use of so-called spacers (components that determine the porosity of alloys produced by powder metallurgy through the formation of gases), which is advantageous since residual spacers inside the materials can negatively influence the biocompatibility of the implants.

Furthermore, it is possible to add, to the powder mixture, metal salts that do not degrade during the formation of the alloy, remain in the alloy, and have a friction-minimizing effect by minimizing the pressing forces required during pressing and during hot working. Examples of such metal salts are, for example, lithium stearate, calcium stearate, and magnesium stearate.

In a preferred embodiment, the powder mixture proposed here has the above-listed components, the first metal powder having a proportion of 40-95 weight %, the second metal powder a proportion of 2-60 weight %, the at least one metal salt powder a proportion of 4-15 weight %, the powder made of a bioresorbable metal alloy a proportion of 0-25 weight %, and a powder from the group of the calcium phosphates a proportion of 0-10% weight %, all components adding up to 100 weight %.

The optional proportion of at least one powder from the group of the calcium phosphates is especially preferred, since calcium phosphates are not only bioresorbable, but rather also represent the bone growth-promoting compounds. This is especially advantageous for use in orthopedic implants.

In a preferred embodiment, the powder mixture proposed here comprises or consists of
45 to 85 weight % magnesium;
8 to 48 weight % of at least one second metal powder selected from the group consisting of aluminum, zinc, and iron;
2 to 8 weight % of a calcium salt powder, preferably calcium phosphate; and
2 to 8 weight % of a zinc, iron, or magnesium salt powder, all components preferably adding up to 100 weight %.

In another embodiment, the powder mixture proposed here comprises or consists of
40 to 55 weight % of magnesium;
35 to 48 weight % of at least one second metal powder selected from the group consisting of zinc and iron;
2 to 8 weight % of a calcium salt powder, preferably calcium phosphate; and
2 to 8 weight % of a zinc, iron, or magnesium salt powder, all components preferably adding up to 100 weight %.

A preferred embodiment of the powder mixture comprises or consists of 30-60 weight % Zn, 30-60 weight % Mg, 10-20 weight % tricalcium phosphate and 10-20 weight % zinc pyrophosphate for production of a bone implant by powder metallurgy.

Another preferred embodiment of the powder mixture comprises or consists of 3-12 weight % Al, 80-90 weight % Mg, 3-10 weight % tricalcium phosphate, 3-5 weight % zinc pyrophosphate and 3-5 weight % of Ca for production by powder metallurgy of a vascular implant.

To ensure optimal alloy formation, it is preferred that the powder of the powder mixture have a average particle size of <45 μm. It is especially preferred that the particles of the first metal powder have a particle size of <35 μm.

If particles, especially of the first metal powder <35 μm are put into the powder mixture, it is possible to avoid significant disadvantages. The disadvantages that can be listed include, for example, that powder with larger particles in the final products leads to local areas that have a uniform composition. The negative consequences of this are both locally different material properties, and also inert degradation behavior, since for example chemically more noble alloy components remain longer as a “skeleton”. An inventive powder with the preferred mean particle sizes allows a well-mixed final product with relatively uniform mechanical and degradation properties.

Introducing and homogeneously distributing powder getter materials such as Ca (in the form of a metal powder) or Ca compounds (in the form of metal salt components) into the starting mixture allows complete or partial reduction of the oxide on the surface of the powder of, for example Mg, Al, and Zn powders during the subsequent thermomechanical treatment. This makes it possible to exert selective influence on alloy effects such as, such as, for example, mass transfer and diffusion of the alloy components. This means that depending on the thickness of the remaining oxide layers on the particles and the temperature, either alloys form between the individual metals (material bonding) (A) or interlocking (B) predominates due to unmelted oxide-coated powder particles of different chemical composition being pressed together. Mixed forms of (A) and (B) are possible. If (B) predominates, a structure interspersed with micropores arises. This does have disadvantages for the mechanical properties, as determined in the tensile test, of the semi-finished product/material produced in this way. However, from these semi-finished products it is possible to produce absorbable implants that can subsequently be infiltrated with various active ingredients. This allows a broad spectrum of medical implants for orthopedics and vascular surgery.

Here additives such as calcium phosphate or zinc pyrophosphate even exhibit an especially advantageous double effect. On the one hand, they have a friction-minimizing effect during the hot working, and on the other hand they have, depending on their proportion, an essential positive effect during the later degradation process. This can consist, e.g., of orthopedic implants growing into the bone matrix more quickly.

Thus, in summary it can be said that the metal salt components in the material that has been produced or is being produced have, in particular, the following functions and advantages:

a) Having an effect as inner friction-minimizing components, both in the powder mixture and also in the semi-finished product for the subsequent steps of pressing or hot working, which obviate the need to apply organic lubricants to the outside.

b) Additionally providing bioresorbable compounds which actively influence cell growth (e.g., in the case of absorbable orthopedic implants).

c) Influencing the degradation behavior of the metal components by releasing compounds inhibiting the progression of corrosion that arises when the metal components come into contact with body fluids.

The use of zinc is advantageous, since zinc has a lower degradation rate than magnesium. This has the consequence that an alloy of zinc and magnesium, and consequently an implant made of such an alloy, has a prolonged life. Furthermore, zinc counteracts restenosis in vascular implants. The presence of zinc reduces the formation of hydrogen, which is released during the degradation of magnesium. In addition, zinc counteracts hemolysis.

Another aspect of this invention relates to a powder metallurgy process for producing a material made of a bioresorbable alloy, the process having at least the steps:
Preparing a preferably homogeneous powder mixture as proposed here;
Pressing the preferably homogeneous powder mixture to produce a semi-finished product (preform);
Hot working the semi-finished product (preform) to produce a material made of a bioresorbable alloy.

The inventive process advantageously allows the production of materials, especially those which cannot be produced by metallurgical processes that involve smelting due to limited solubility and especially those which are characterized by a comparatively wide range of mechanical and biochemical properties. An essential feature of the production process that is used is the combination of absorbable metal matrix materials with one or more bioactive substances, which are put together in a semi-finished product by material bonding or interlocking. The inventive solution further consists of exploiting the special temperature-dependent material properties of each individual component so that the production process produces from them material compounds with mechanical load-bearing capability and made-to-order biological properties.

Varying not only the chemical composition, but rather also the process parameters for pressing allows variations in the semi-finished product structure with respect to the strength properties, the plasticizing property, and the porosity. This makes it possible to produce implants that are made-to-order for the later use.

In comparison with conventional powder metallurgy, the process proposed here is able to eliminate the hot sintering process that is used for the production of preforms (semi-finished product). Moreover, the use of a hot working step (e.g., in the form of an extrusion process) allows wide variation with respect to the material density/porosity. This opens a wide spectrum of possible applications, ranging from bone surgery through plastic reconstruction of skull defects all the way to vascular surgery.

Furthermore, in comparison to conventional powder metallurgy processes metal powders consisting of the respective metal element are provided for forming the respective powder mixture as used herein. Starting from the powder mixture as described here an alloy is formed by the inventive process as described herein. In contrast, ion conventional powder metallurgy processes alloy powders are provided as starting materials and are further processed to form alloys—again. Hence, initially an alloy was formed by conventional smelting which is then further alloyed by powder metallurgy. The precursors for the powder metallurgic steps are alloys. The present invention however provides for powder metallurgic processes by capable of forming alloys from element powders and the initial alloying is achieved during the inventive process.

One embodiment of the inventive process provides that the powder mixture have aluminum (preferably pure Al 99.9%) and magnesium (preferably pure Mg 99.9%) in the form of metal powders and tricalcium phosphate and zinc pyrophosphate in the form of metal salt powders.

Furthermore, one embodiment of the inventive process provides that the powder mixture have zinc (preferably pure Zn 99.9%) and magnesium (preferably pure Mg 99.9%) in the form of metal powders and tricalcium phosphate and zinc pyrophosphate in the form of metal salt powders.

Furthermore, one embodiment of the inventive process provides that the powder mixture have iron (preferably pure Fe 99.9%) and magnesium (preferably pure Mg 99.9%) in the form of metal powders and calcium fluoride and zinc pyrophosphate in the form of metal salt powders.

Furthermore, one embodiment of the inventive process provides that the powder mixture have zinc (preferably pure Zn 99.9%) and magnesium (preferably pure Mg 99.9%) in the form of metal powders and calcium fluoride and magnesium carbonate in the form of metal salt powders.

Furthermore, one embodiment of the inventive process provides that the powder mixture have zinc (preferably pure Zn 99.9%) in the form of a metal powder and calcium fluoride and magnesium carbonate in the form of metal salt powders and an AlMg alloy, preferably with about 68 weight % Mg (preferably having purity of 99.9%) in the form of a powder of a bioresorbable metal alloy.

The preparation of the powder mixture preferably succeeds if the components are put together and homogenized, that is mixed and distributed. Suitable homogenization succeeds, for example, by using a ball mill.

A preferred embodiment of the inventive process provides that the semi-finished product (preform) is heated and placed under pressure in such a way to form a metal alloy that has at least two of the metal components. Furthermore, it is advantageous to heat the powder mixture quickly and then press it immediately, since this makes it possible to avoid undesired segregation phenomena in the preform.

Furthermore, a preferred embodiment of the inventive process provides that the semi-finished product be worked by heating, in particular by extruding the semi-finished product. Here it is further advantageous that the melting point of at least one of the alloy components be exceeded, at least in the short-term. Exceeding and fusing or melting one of the alloy components strongly improves the material bonding within the material.

Relating to this, the addition of zinc is preferred, since zinc has a relatively low melting point of 419.5° C. During the hot working, local molten Zn-rich phases are already produced over the entire cross section of the semi-finished product. These phases cover all other components that remain in the solid state at the applied temperature (that is, whose melting point is not exceeded). This generates a cohesion of all powder components with mechanical load-bearing capability.

Furthermore, compositions with magnesium and aluminum powder mixtures are advantageous, since such combinations allow a reduction in the melting point to 437° C.

It is also possible to achieve a melting point reduction by the optional addition of eutectic alloys, for example a ZnMg alloy with a eutectic temperature of 341° C.

A preferred embodiment of the inventive process further provides that the powder mixture be pressed into the semi-finished product with a pressure in the range of 1 kPa to 20 kPa.

A preferred embodiment of the inventive process further provides that when the semi-finished product is heated, it is heated to a temperature in the range of 200° C. to 500° C., preferably in the range from 250° C. to 450° C.

Furthermore, it is preferred that for hot working, the semi-finished product be heated to a temperature in the range from 200° C. to 500° C., preferably in the range from 250° C. to 450° C., and that an extrusion pressure of 1 kPa to 20 kPa, preferably of 2 to 10 kN be used.

Furthermore, a preferred embodiment of the inventive process provides that the pressing of the powder mixture shapes it into a bar-shaped, cylindrical, or hollow cylindrical semi-finished product. Such geometric shapes have the advantage that they allow very good hot working, in particular extrusion. This has a positive effect on the economics of the process.

In one embodiment of the process proposed here, the semi-finished product (the preform) is inductively heated after the pressing. This has the advantage that, on the one hand, any inner tensions there might be in the semi-finished product (preform) are minimized, and on the other hand the shorter warm-up time avoids material segregations in the preform.

In another embodiment of the process proposed here, a step follows in which the material made of a bioresorbable alloy is hot worked and then shaped into a medical implant.

Furthermore, a preferred embodiment of the inventive process provides that the material made of a bioresorbable alloy be shaped by machining or non-cutting shaping processes into medical implants.

Another aspect of this invention relates to a material that has been produced by the inventive process.

Furthermore, this invention relates to a process for producing a medical implant, this process having the steps of an inventive process for producing the material and forming, from the semi-finished product produced in this way, a medical implant, in particular by machining the material (e.g., turning it on a lathe) and/or processing it by non-cutting processes (e.g., pickling or electropolishing).

The implant can be, e.g., one of the following implants: an intramedullary nail, a bone plate, a vascular implant, or a stent.

In particular, an implant is proposed whose base body and surface are polymer-free, porous, and elute an active ingredient. The surface in question here is the surface of the implant body itself, not an applied porous surface, which is applied by a coating, for example. The porosity of the surface and of the base body results from the implant body's overall porosity, which is controlled by the addition of the metal salt components that are considered suitable here, but cannot be completely prevented. The active ingredient is incorporated in the pores resulting from the process. Thus, selection of the correct initial components for the powder mixture proposed here can also indirectly determine the depth and volume of the pores, which function as active ingredient reservoirs, and thus also the amount of the active ingredient and the depth of the active ingredient in the implant. Thus, it is possible to prepare implants whose active ingredient charge can be safely delivered, almost without losses, to the site of action, and implanted there, and whose potential effect can be predetermined by the manufacturing conditions and parameters. According to the invention, the presence of pores is realized in such a way that an active ingredient charge is possible and that the mechanical properties of the implants are maximally maintained in comparison with non-porous implants. In particular, this is achieved by the possibility of doing without spacer substances.

### DETAILED DESCRIPTION

The discussion below is intended to explain other features and advantages of this invention on the basis of individual embodiments or examples of the invention.

### Example 1

A powder mixture made of 10.98 g pure Al (99.9%) and 89.02 g pure Mg (99.9%) is weighed out and homogenized in a ball mill at 3,000 rpm. To these 100 g of powder, 10 g of another powder mixture are added. This powder mixture consists of 5.00 g of tricalcium phosphate and 5.00 g of zinc pyrophosphate. This second powder mixture was previously homogenized under the same conditions. This powder mixture that now consists of 4 components is filled into a hollow cylindrical pressing device which has a diameter of 5.00 mm and a depth of at least 26 mm. The bottom of this hollow cylinder is tightly closed by a suitable device. The filling height of the powder mixture is about 10 mm. Following that, a slightly undersized die with a diameter of 4.99 mm is introduced, and the powder mixture is pressed at room temperature and a pressure force of about 3 kN. This reduces the original 10 mm height of packing of the powder mixture to about 6 mm. After the bottom of the hollow cylinder is opened, it is now possible to press the pressed preform out using the same die. This cylindrical blank is then processed into a hollow cylinder by shaping processes that involve machining. The resulting inside diameter is 1.6 mm. In the next step, this hollow cylinder is put on a hard metal die whose outside diameter is about 1.58 mm. The hard metal die with the blank put on it is pushed so that it is centered into a matrix that has been preheated (e.g., by means of induction) to 280° C., and the blank is shaped, with a pressure force of about 5 kN, into a miniature tube. This tube with outside diameters between 2.0 and 3.00 mm can then undergo further processing to shape it into a wide range of absorbable implants such as cannulated Kirschner wires, surgical screws, or stents for coronary but also peripheral applications. The degradation time of such implants is between 2 months and 18 months, depending on the wall thickness profile.

### Example 2

A powder mixture made of 47.04 g pure Zn (99.9%) and 52.96 g pure Mg (99.9%) is weighed out and homogenized in a ball mill at 3,000 rpm. The further steps are the same as in example 1, except that the temperature used is 325° C. and the pressure force is 7 kN.

### Example 3

A powder mixture made of 47.04 g pure Fe (99.9%) and 52.96 g pure Mg (99.9%) is weighed out and homogenized in a ball mill at 3,000 rpm. To these 100 g of powder, 10 g of another powder mixture are added. This powder mixture consists of 5.00 g of calcium fluoride and 5.00 g of zinc pyrophosphate. This third powder mixture was previously homogenized under the same conditions. This powder mixture that now consists of 4 components is filled into a hollow cylindrical pressing device which has a diameter of 5.00 mm and a depth of at least 26 mm. The bottom of this hollow cylinder is tightly closed by a suitable device. The filling height of the powder mixture is about 10 mm. Following that, a slightly undersized die with a diameter of 4.99 mm is introduced, and the powder mixture is pressed at room temperature and a pressure force of about 3 kN. This reduces the original 10 mm height of packing of the powder mixture to about 7 mm. After the bottom of the hollow cylinder is opened, it is now possible to press the pressed preform out using the same die. This cylindrical blank then undergoes machining processes to shape it into a hollow cylinder. The resulting inside diameter is 1.6 mm. In the next step, this cylindrical blank is put on a hard metal die whose outside diameter is about 1.58 mm. The hard metal die with the blank put on it is pushed so that it is centered into a matrix that has been preheated (e.g., by means of induction) to 420° C., and the blank is shaped, with a pressure force of about 7 kN, into a miniature tube. This tube test with outside diameters between 2.0 and 3.00 mm can then undergo further processes to shape it into a wide range of absorbable implants such as cannulated Kirschner wires, surgical screws, or stents for coronary but also peripheral applications. The degradation time of such implants is between 2 months and 18 months, depending on the wall thickness profile.

### Example 4

A powder mixture made of 47.04 g pure Zn (99.9%) and 52.96 g pure Mg (99.9%) is weighed out and homogenized in a ball mill at 3,000 rpm. To these 100 g of powder, 10 g of another powder mixture are added. This powder mixture consists of 5.00 g of calcium fluoride and 5.00 g of magnesium carbonate. This second powder mixture was previously homogenized under the same conditions. The further steps are the same as in example 1, except that the temperature used is 325° C. and the pressure force is 7 kN.

1. A powder mixture for production of an alloy by powder metallurgy, the powder mixture comprising:
a first metal powder selected from the group consisting of magnesium, aluminum, zinc, calcium, and iron;
at least one second metal powder different from the first metal powder, the at least one second metal powder selected from the group consisting of magnesium, aluminum, zinc, calcium, and iron;
at least one metal salt powder; and
optionally at least one powder of a bioresorbable, eutectic metal alloy.

2. The powder mixture according to claim 1, wherein the first metal powder is magnesium.

3. The powder mixture according to claim 1, wherein the at least one metal salt powder is selected from the group consisting of magnesium hydrogen phosphate, magnesium carbonate, calcium phosphate, tricalcium phosphate, calcium carbonate, calcium hydroxide, calcium fluoride, lithium stearate, calcium stearate, magnesium stearate, zinc pyrophosphate, and zinc carbonate.

4. The powder mixture according to claim 1, wherein the first metal powder has a proportion of 40-95 weight %, the second metal powder a proportion of 2-60 weight %, the metal salt powder a proportion of 4-15 weight %, and the powder made of a bioresorbable metal alloy a proportion of 0-25 weight %, all components adding up to 100 weight %.

5. The powder mixture according to claim 1, wherein the particles of the first metal powder have a particle size of <45 μm.

6. A powder metallurgy process for producing a material from a bioresorbable alloy, the process comprising the steps of:
preparing the powder mixture according to claim 1;
pressing the powder mixture to produce a semi-finished product;
hot working the semi-finished product to produce a material made of a bioresorbable alloy.

7. The process according to claim 6, wherein the step of hot working the semi-finished product is done by extrusion.

8. The process according to claim 6, wherein the powder mixture is pressed, with a pressure in the range from 1 kPa to 20 kPa, into the semi-finished product.

9. The process according to claim 6, wherein the semi-finished product is heated to a temperature in the range from 200° C. to 500° C. during the hot working.

10. The process according to claim 6, wherein the material made of the bioresorbable alloy is shaped into a medical implant.

11. A material produced by the process according to claim 6.

12. A bioresorbable medical implant that is produced according to claim 10.

13. A bioresorbable medical implant whose base body and surface are polymer-free, porous, and active ingredient-eluting.

14. The implant according to claim 13, wherein at least one active ingredient is located in the pores of the surface of the implant.

15. The implant according to claim 14, the implant being selected from the group consisting of an intramedullary nail, a bone plate, a vascular implant, and a stent.
